(19) United States
(12) Patent Application Publication
(10) Pub. No.: US 2002/0193593 A1
(43) Pub. Date: Dec. 19, 2002

(54) Amine salts free from amino-aromatic moieties that inhibit corrosion during pickling as effectively as prior art amine salts with amino-aromatic moieties

(76) Inventor: James P. Bershas, West Bloomfield, MI (US)

Correspondence Address: Stephen D. Harper, Law Department, Suite 200, 2500 Renaissance Blvd., Gulph Mills, PA, 19406, US

(21) Appl. No.: 10/109,345

(22) Filed: Mar. 28, 2002

Related U.S. Application Data
(60) Provisional application No. 60/279,551, filed on Mar. 29, 2001.

(51) Int. Cl. C07D 265/28; C07C 225/06

(52) U.S. Cl. 544/170; 564/502

(57) ABSTRACT

Disclosed herein are secondary and/or tertiary amine salts free from amino-aromatic moieties that effectively inhibit corrosion during pickling. Said amine salts are prepared by one or more reactions among the following reactants:

(A) formaldehyde;

(B) at least one ketone conforming to the immediately following general formula

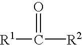

wherein each of R¹ and R², which may be the same or different, represents a monovalent organic moiety that does not include an amino-aromatic ring moiety;

(C) at least one amine constituted of molecules each of which includes at least one —N—H moiety and at least one ether or hydroxyl oxygen atom but does not include an amino-aromatic ring moiety; and

(D) an acid that is not part of any one of reactants (A) through (C) as described immediately above

to produce at least one secondary or tertiary amine salt reaction product in which the hydrogen atom in said at least one —N—H moiety has been replaced by a moiety having one of the two immediately following general formulas

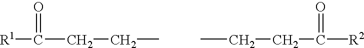

wherein each of R¹ and R² has the same meaning as in the description of reactant (B) above.

### BACKGROUND OF THE INVENTION

In the well-established industrial process called “pickling”, metal oxides and/or other materials, often forming heavy scales, are removed from metal surfaces by exposing these surfaces to strong acids. In order to minimize unwanted dissolution of elemental metal from the surfaces along with the oxides and/or other materials that it is desired to remove, it has long been customary in the art to use pickling inhibitors. The most effective of these in current and prior art are complex amine salts that include at least one amino-aromatic ring moiety.

All compounds containing amino-aromatic rings are widely regarded as suspect carcinogens, and the process of manufacturing the pickling inhibitors now considered most effective is therefore considered possibly hazardous to health of the manufacturing workers unless expensive precautions are taken. Accordingly, a major object of this invention is to provide pickling inhibitors that do not contain amino-aromatic ring moieties but are as effective in inhibition as the best prior art amine salts that do contain amino-aromatic rings. Other alternative and/or concurrent and/or more detailed objects of the invention will be apparent from the further description below. Except in the claims and the specific examples, or where otherwise expressly indicated, all numerical quantities in this description indicating amounts of material or conditions of reaction and/or use are to be understood as modified by the word “about” in describing the broadest scope of the invention. Practice within the numerical limits stated is generally preferred, however. Also, throughout this specification unless expressly stated to the contrary: percent, “parts of”, and ratio values are by weight; the term “polymer” includes “oligomer”, “copolymer”, “terpolymer”, and the like; the first definition or description of the meaning of a word, phrase, acronym, abbreviation or the like applies to all subsequent uses of the same word, phrase, acronym, abbreviation or the like and applies, mutatis mutandis, to normal grammatical variations thereof; the term “mole” means “gram mole” and “mole” and its variations may be applied to ions, moieties, elements, and any other actual or hypothetical entity defined by the number and type of atoms present in it, as well as to materials with well defined neutral molecules; the description of a group or class of materials as suitable or preferred for a given purpose in connection with the invention implies that mixtures of any two or more of the members of the group or class are equally suitable or preferred; description of preparation of compositions or components thereof by utilizing electrically neutral chemical constituents refers to the constituents at the time of first addition to any combination specified in the description, and does not necessarily preclude chemical interactions among the constituents of a mixture, or physical changes in such properties as dispersed particle sizes and distribution of materials between dispersed and continuous phases in a dispersion, after mixing has occurred; specification of materials in ionic form implies the presence of sufficient counterions to produce electrical neutrality for the composition as a whole; and any counterions thus implicitly specified preferably are selected from among other constituents explicitly specified in ionic form, to the extent possible; otherwise such counterions may be freely selected, except for avoiding counterions that act adversely to the objects of the invention.

### BRIEF SUMMARY OF THE INVENTION

It has been found that the above stated major object of the invention is achieved by secondary and/or tertiary amine salts and mixtures of such amine salts that are prepared by at least one reaction among the following reactants:

(A) formaldehyde;

(B) at least one ketone conforming to the immediately following general formula

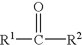

wherein each of R¹ and R², which may be the same or different, represents a monovalent organic moiety that does not include an amino-aromatic ring moiety;

(C) at least one amine constituted of molecules each of which includes at least one —N—H moiety and at least one ether or hydroxyl oxygen atom but does not include an amino-aromatic ring moiety; and

(D) an acid that is not part of any one of reactants (A) through (C) as described immediately above

to produce at least one secondary or tertiary amine salt reaction product in which the hydrogen atom in said at least one —N—H moiety has been replaced by a moiety having one of the two immediately following general formulas

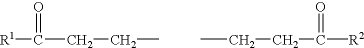

wherein each of R¹ and R² has the same meaning as in the description of reactant (B) above. If amine reactant (C) as described above includes molecules having an —NH₂ moiety, which is considered herein to be a particular example of an —N—H moiety, either or both of hydrogen atoms in this moiety may be replaced as described above in any molecule of the reaction product. Also, if amine reactant (C) as described above includes molecules having more than one —N—H moiety, a hydrogen atom in either or both of these moieties may be substituted as described above in any molecule of the reaction product.

There may be more than one reaction stage among reactants of the types (A) through (D) as described above to produce a final product amine salt or mixture of amine salts according to the invention, and if there is more than one such reaction, the chemical nature of reactants (B) through (D) in any subsequent one of such reactions may be identical to or different from those used in the first such reaction, so long as they remain within the scope of the definitions of reactants (B) through (D) as described above. If there is more than one reaction stage in a process of producing a mixture of amine salts according to the invention, reactants for the first reaction may designated (B.1) through (D.1), those for the second reaction may be designated (B.2) through (D.2), and those for any subsequent reaction are designated similarly with a cardinal number corresponding to the ordinal number of the reaction. Furthermore, in reaction stages after the first, from one to three of reactants of types (A) through (D) may not need to be added to the reaction mixture, if sufficient amounts of suitable reactants of the non-added types remain in the reaction mixture to produce further chemical reactions therein.

In addition to the amine salts themselves, other embodiments of the invention include pickling inhibitor concentrates that contain such amine salts together with at least one auxiliary inhibitor such as thiourea and acetylene alcohols, pickling solutions containing inhibition-effective concentrations of these amine salts, processes for making the amine salts, and processes for using the pickling solutions containing inhibition-effective concentrations of these amine salts.

### DETAILED DESCRIPTION OF THE INVENTION

For convenience and economy, formaldehyde is normally preferably supplied to a process according to this invention by an aqueous solution, only the actual formaldehyde content of any such solution being considered in determining any amount or ratio of this material specified below. However, any other source of formaldehyde that does not simultaneously introduce materials detrimental to the desired chemical reactions may alternatively be used.

The following preferences apply for at least, with increasing preference in the order given, 50, 60, 70, 75, 80, 85, 90, 95, or 99 number percent of the molecules of reactant (B) as described above in a first or sole reaction to produce an amine salt or mixture of amine salts according to the invention, each preference being individually preferred and combinations of the preferences being still more preferred, the degree of preference increasing with the number of preferences so combined:

at least one, more preferably exactly one, of R¹ and R² includes an aromatic ring moiety, which may or may not be substituted in any position other than that required to connect the aromatic ring moeity to the carbonyl group in the ketone molecule;

if one of R¹ and R² includes an aromatic ring moiety:

the ring preferably is a phenyl ring;

at least for economy, the ring preferably is unsubstituted; and

the number of carbon atoms in the molecule preferably is at least, with increasing preference in the order given, 4, 5, 6, 7, or 8 and independently preferably is not more than, with increasing preference in the order given, 20, 18, 16, 14, 12, 10, 9, or 8.

The following preferences apply for at least, with increasing preference in the order given, 50, 60, 70, 75, 80, 85, 90, 95, or 99 number percent of the molecules of reactant (C) as described above in a first or sole reaction to produce an amine salt or mixture of amine salts according to the invention, each preference being individually preferred and combinations of the preferences being still more preferred, the degree of preference increasing with the number of preferences so combined:

each molecule contains not more than, with increasing preference in the order given, 4, 3, 2, or 1 nitrogen atom(s);

each molecule contains not more than, with increasing preference in the order given, 4, 3, 2, or 1 oxygen atom(s);

at least for economy, each molecule contains no atoms other than carbon, hydrogen, oxygen, and nitrogen atoms;

for at least, with increasing preference in the order given, 20, 30, 40, 45, 50, 52, 54, or 56 number percent of the molecules but for not more than, with increasing preference in the order given, 99, 95, 90, 85, 80, 75, 70, 65, 63, 61, 59, or 57 number percent of the molecules:

each molecule includes a 5- or 6-membered ring of atoms selected from the group consisting of carbon, oxygen, and nitrogen;

at least one, or more preferably at least both, of an oxygen atom and a nitrogen atom is a member of a 5- or 6-membered ring within the molecule;

any oxygen atom present within the molecule is directly bonded to two distinct carbon atoms;

there are not more than, with increasing preference in the order given, 2, 1, or 0 carbon-carbon double bonds in the molecule; and

the number of carbon atoms in the molecule is at least, with increasing preference in the order given, 3 or 4 and independently preferably is not more than, with increasing preference in the order given, 20, 16, 14, 12, 10, 8, 7, 6, 5, or 4; and

for at least, with increasing preference in the order given, 10, 20, 30, 35, 37, 39, 41, or 43 number percent of the molecules but for not more than, with increasing preference in the order given, 99, 70, 60, 55, 52, 50, 48, 46, or 44 number percent of the molecules:

there is no ring of carbon, oxygen, and/or nitrogen atoms in the molecule;

there are not more than, with increasing preference in the order given, 2, 1, or 0 carbon-carbon double bonds in the molecule; and

the number of carbon atoms in the molecule is at least, with increasing preference in the order given, 2 or 3 and independently preferably is not more than, with increasing preference in the order given, 10, 9, 8, 7, 6, 5, 4, or 3;

an amino nitrogen atom is directly bonded to a terminal carbon atom in the molecule;

an oxygen atom is directly bonded to a non-terminal carbon atom in the molecule; and

any oxygen atom present is part of a hydroxyl moiety.

In addition to and independently of the preferences given above for reactants (B) and (C) individually, the following ratios between various reactants initially are also preferred for a first or sole reaction to produce an amine salt or mixture of amine salts according to the invention, each preference being individually preferred and combinations of the preferences being still more preferred, the degree of preference increasing with the number of preferences so combined:

the molar ratio of formaldehyde molecules in reactant (A) to amino groups in reactant (B) preferably is at least, with increasing preference in the order given, 0.20:1.00, 0.30:1.00, 0.40:1.00, 0.60:1.00, 0.80:1.00, 1.00:1.00, 1.20:1.00, 1.30:1.00, 1.40:1.00, 1.50:1.00, 1.60:1.00, 1.70:1.00, 1.80:1.00, 1.90:1.00, 1.95:1.00, or 1.99:1.00 and independently preferably is not more than, with increasing preference in the order given, 8:1.00, 6:1.00, 4.0:1.00, 3.5:1.00, 3.0:1.00, 2.5:1.00, 2.3:1.00, or 2.1:1.00 (if there is no objection to high levels of free formaldehyde in the amine salt or mixture of amine salts produced, still higher ratios of formaldehyde to reactant (B) may be satisfactorily used, but normally a maximum concentration 0.1% of free formaldehyde in the amine salt or mixture of amine salts is preferred, to avoid a major odor nuisance, and in such instances the ratios as given above are preferred);

the molar ratio of amino groups in reactant (B) to carbonyl moieties in reactant (C) preferably is at least, with increasing preference in the order given, 0.40:1.00, 0.60:1.00, 0.80:1.00, 1.00:1.00, 1.20:1.00, 1.30:1.00, 1.40:1.00, 1.50:1.00, 1.60:1.00, 1.70:1.00, 1.80:1.00, 1.90:1.00, 2.00:1.00, 2.10:1.00, 2.20:1.00, 2.30:1.00, 2.35:1.00, or 2.39:1.00 and independently preferably is not more than, with increasing preference in the order given, 10:1.00, 8:1.00, 6:1.00, 4.0:1.00, 3.5:1.00, 3.1:1.00, 2.9:1.00, 2.7:1.00, or 2.5:1.00.

The molar ratio of acid in the acid reactant (D) to the moles of amino nitrogen present in reactant (B) preferably is at least, with increasing preference in the order given, 0.70, 0.80, 0.90, 1.00, 1.02, 1.04, or 1.06:1.00 and independently preferably, at least for economy, is not more than, with increasing preference in the order given, 2.0, 1.8, 1.6, 1.4, 1.30, 1.25, 1.20, 1.15, 1.10, or 1.08:1.00.

The ketone (B) must hav at least one hydrogen atom one one of the carbons directly attached to the carbonyl moiety in order for the Mannich Reaction to proceed. Particularly preferred ketones (B) are acetophenone and acetone. Preferred amines (C) are morpholine and 1-amino-2hydroxy propane, and a mixture thereof is particularly preferred.

Preferred acid reactants (D) for a process of making an amine salt or mixture of amine salts according to the invention are selected from the group consisting of hydrochloric acid and hydroxy organic carboxylic acids, most preferably hydroxy acetic acid. If the presence of chloride in the final product is not objectionable, hydrochloric acid is preferred for economy. If very little or no chloride is tolerable, at least one hydroxy organic acid is preferably used as the only catalyst. Mixtures in any proportion of hydrochloric and hydroxy organic acids are technically satisfactory and may be preferred if only slight amounts of chloride in the final product are tolerable. Further particularly preferred acids (D), especially for obtaining low amounts of residual formaldehyde, are glycolic acid or a mixtures of glycolic acid and hydrochloric acid.

A process according to the invention for making an amine salt or mixture of amine salts for use as an inhibitor preferably comprises, more preferably consists essentially of, or still more preferably consists of, the following operations:

(I) providing specified amounts of each of reactants (A) through (D) as described above;

(II) mixing together the amounts of reactants (B) and (C) provided in operation (I), optionally together with a solvent that is not part of any of reactants (A) through (D) to form a liquid first mixture;

(III) emplacing said first mixture in a container provided with:

means for mechanical agitation of its liquid contents,

means for measuring, and optionally also for controlling, the temperature of liquid contained in said container,

means for condensing vapor produced by the contents of the container and returning said condensed vapor to the liquid contents of the container, and

addition-container means for adding a plurality of distinct portions of any liquid contained therein sequentially to said container in a controlled manner;

(IV) emplacing said acid reactant (D) as provided in operation (I) in said addition-container means;

(V) adding said acid reactant (D) from said addition-container means to said container in successive distinct portions in a controlled manner that causes the first of said portions of said acid reactant (D) to mix with said first mixture to form a first first-intermediate mixture, the second of said added portions of said acid reactant (D) to said first first-intermediate mixture to form a second first-intermediate mixture, and the like, until all of said portions have been added to form a final first-intermediate mixture, with a sufficient time interval between each two successive said additions of portions of said acid reactant (D) that:

at least, with increasing preference in the order given, 90, 92, 94, 96, 98.0, 98.5, 99.0, 99.5, or 99.9 percent of the vapor expelled from the liquid contents of said container is returned thereto as liquid by said means for condensing vapor and returning condensed vapor; and

the temperature of the contents of said container does not exceed, with increasing preference in the order given, 95, 90, 85, 80, or 75° C.;

(VI) emplacing within said addition-container the amount of reactant (A) provided in operation (I);

(VII) adding said reactant (A) from said addition-container means to said container in successive distinct portions in a controlled manner that will cause the first of said portions of said reactant (A) to mix with said final first-intermediate mixture to form a first second-intermediate mixture, the second of said added portions of said reactant (A) to said first second-intermediate mixture to form a second second-intermediate mixture, and the like until all of said portions of reactant (A) have been added to form a final second-intermediate mixture, with a sufficient time interval between each two successive said additions of portions of said acid reactant (D) that:

at least, with increasing preference in the order given, 90, 92, 94, 96, 98.0, 98.5, 99.0, 99.5, or 99.9 percent of the vapor expelled from the liquid contents of said container is returned thereto as liquid by said means for condensing vapor; and

the temperature of the contents of said container does not exceed, with increasing preference in the order given, 95, 90, 85, 80, or 75° C.;

(VIII) maintaining said final second-intermediate mixture at a temperature that is at least, with increasing preference in the order given, 60, 65, 70, 75, or 80° C. and independently preferably, at least for economy, is not more than 95° C. for a time interval that is at least, with increasing preference in the order given, 8, 12, 16, 20, or 24 hours and independently, at least for economy, preferably is not more than, with increasing preference in the order given, 72, 60, 48, 36, or 25 hours to produce an aged second-intermediate mixture, sufficient condensing capacity being maintained during this interval that at least, with increasing preference in the order given, 90, 92, 94, 96, 98.0, 98.5, 99.0, 99.5, or 99.9 percent of the vapor expelled from the liquid contents of said container is returned thereto as liquid by said means for condensing vapor; and, optionally, one or more of the following operations;

(IX) cooling said aged second-intermediate mixture to a temperature that is not more than, with increasing preference in the order given, 75, 70, 65, 60, 55, or 50 to form a cooled second-intermediate mixture;

(X) mixing with said aged second-intermediate mixture or with said cooled second-intermediate mixture a quantity of a reactant (B.2) as described above to form a third-intermediate mixture;

(XI) emplacing within said addition-container a quantity (A′) of formaldehyde;

(XII) adding said quantity (A′) from said addition-container means to said container in successive distinct portions in a controlled manner that will cause the first of said portions of said quantity (A′) to mix with said third-intermediate mixture to form a first fourth-intermediate mixture, the second of said added portions of said quantity (A′) to said first fourth-intermediate mixture to form a second fourth-intermediate mixture, and the like until all of said portions have been added to form a final fourth-intermediate mixture, with a sufficient time interval between each two successive said additions of portions of said quantity (A′) that:

at least, with increasing preference in the order given, 90, 92, 94, 96, 98.0, 98.5, 99.0, 99.5, or 99.9 percent of the vapor expelled from the liquid contents of said container is returned thereto as liquid by said means for condensing vapor; and

the temperature of the contents of said container does not exceed, with increasing preference in the order given, 80, 75, 70, 65, or 60° C.;

(XIII) maintaining said final fourth-intermediate mixture at a temperature that is at least, with increasing preference in the order given, 40, 45, 50, 55, or 60° C. and independently preferably, at least for economy, is not more than, with increasing preference in the order given, 80, 75, 70, 65, or 60° C., for a time interval that is at least, with increasing preference in the order given, 8, 12, 16, 20, or 24 hours and independently, at least for economy, preferably is not more than, with increasing preference in the order given, 72, 60, 48, 36, or 25 hours to produce an aged fourth-intermediate mixture, sufficient condensing capacity being maintained during this interval that at least, with increasing preference in the order given, 90, 92, 94, 96, 98.0, 98.5, 99.0, 99.5, or 99.9 percent of the vapor expelled from the liquid contents of said container is returned thereto as liquid by said means for condensing vapor and returning condensed vapor; and

(XIV) separating from:

said aged second-intermediate mixture;

said aged fourth-intermediate mixture; or

an aged sixth-, eighth-, or other ordinally even numbered-intermediate mixture prepared by iteration of operations (X) through (XIII) as described above, with all ordinal numbers in operations (X) through (XIII) being increased by one for the first such iteration, by two for the second such iteration, and the like

all constituents thereof that vaporize therefrom at normal atmospheric pressure at a temperature of 90° C., to produce a volatiles-stripped mixture that constitutes said amine salt or mixture of amine salts for use in inhibition according to the invention.

Preferably all of optional operations (IX) through (XIV) as described above are included in a process according to the invention for making an amine salt or mixture of amine salts useful for inhibition, and even if operations (IX) through (XIII) as described above are not included in such a process, operation (XIV) as described above nevertheless preferably is included. If operation (X) is included, operations (XI) and (XII) at least should also be included. At least for economy, there preferably are no iterations of operations (X) through (XIII).

When operations (X) through (XII) are included in a process according to the invention for making an amine salt or mixture of amine salts useful for inhibition, the following preferences apply, each preference being individually preferred and combinations of the preferences being still more preferred, the degree of preference increasing with the number of individual preferences so combined:

at least, with increasing preference in the order given, 50, 60, 70, 75, 80, 85, 90, 95, or 99 number percent of the molecules of reactant (B.2) are selected from ketones that contain not more than, with increasing preference in the order given, 8, 7, 6, 5, 4, or 3 carbon atoms;

the ratio of the number of moles of carbonyl moieties in reactant (B.2) to the number of moles of carbonyl moieties in reactant (B) is at least, with increasing preference in the order given, 1:1.00, 3:1.00, 5:1.00, 6.0:1.00, 7.0:1.00, 7.5:1.00, 8.0:1.00, 8.5:1.00, or 9.0:1.00 and independently preferably is not more than, with increasing preference in the order given, 50:1.00, 40:1.00, 30:1.00, 25:1.00, 20:1.00, 18:1.00, 16:1.00, 14:1.00, or 12:1.00; and

the ratio of the number of moles of carbonyl moieties in reactant (B.2) to the number of moles of formaldehyde in quantity (A′) is at least, with increasing preference in the order given, 0.5:1.00, 1.0:1.00, 1.5:1.00, 2.0:1.00, 2.5:1.00, 3.0:1.00, or 3.5:1.00 and independently preferably is not more than, with increasing preference in the order given, 20:1.00, 15:1.00, 10:1.00, 8:1.00, or 6:1.00.

Although the material in reactant (B.2) is designated as a reactant, it will be apparent to those skilled in the art that not all of the preferred amounts specified above can actually react. The “excess” amount of ketone is believed to function instead as a solvent or hydrotroping agent, maintaining the homogeneity of the mixtures during intermediate stages of reaction. Any amount of unreacted ketone added as part of reactant (B.2) normally will be separated as volatile matter in operation (XIV) as described above.

In a process for using an amine salt or mixture of amine salts according to the invention, a mass of the amine salt or mixture of amine salts is preferably substituted for an equal mass of amine salt or mixture of amine salts that contain amino-aromatic ring moieties. It is particularly advantageous and therefore preferred to substitute an amine salt or mixture of amine salts according to the invention for a salt or mixture of salts of 1,3,5-triazine, hexahydro-1,3,5-tris(2-methylphenyl), the trihydrochloride of which is a very widely used primary inhibitor component of commercial pickling inhibitors.

The amine salts described herein have been found to be effective corrosion inhibitors and are of particular value as inhibitors in acid mediums or baths for the prevention or retardation of the attack of such baths upon metal surfaces and especially upon steel surfaces. The amine salts are highly useful for the protection of metals generally and particularly for the protection of ferriferous metals during the removal of water-borne deposits, of heat scale and of rust where the removal is carried out using acids such as hydrochloric, sulfuric, phosphoric, acetic, formic and the like.

The composition of the pickling or metal treating solutions containing the amine salts, their temperature of use and other factors vary depending upon the metal to be treated and the type of deposit to be removed. However, the primary function of such solutions is to remove undesirable deposits from the metal surface while at the same time minimizing or restraining the attack of the acid upon the exposed metal itself. Both acid and metal are thereby conserved and damage such as embrittlement, burning or overpickling is prevented or reduced by use of the amine salt. The quantity of the inhibitor or mixture of inhibitor to be used will, of course, vary depending upon any given set of conditions, but those skilled in the art of pickling can easily determine by experimentation the optimum proportions to be employed. Under certain conditions, as little as 0.01 weight % amine salt in the acid pickling bath will reduce the amount of metal corrosion as compared to an acid pickling bath which is free of amine salt.

The amine sales of this invention may be used in combination with any of the other additives or components conventionally employed in the acid pickling art including, for example, other types of inhibitors, wetting agents, and the like.

The invention may be further appreciated by consideration of the following working examples, which are not to be construed as limiting the invention in any way except to whatever extent limitations from the examples may appear in the appended claims.

Group 1

### AMINE SALT MIXTURE SYNTHESIS EXAMPLE 1.1

A three necked 1000 milliliter (this unit being hereinafter usually abbreviated as “ml”) round bottom flask was equipped with a mechanical stirrer, a type J thermocouple attached to a temperature controller and monitor, a reflux condenser, and a 125 ml pressure-equalized dropping funnel. The flask was charged sequentially with morpholine (43.6 grams, this unit being hereinafter usually abbreviated as “g”, which equals 0.501 moles), 1-amino-2-hydroxy propane (28.2 g, 0.376 moles), acetophenone (43.8 g, 0.365 moles). The cooling water hooked up to the condenser was turned on and the dropping funnel was charged with a mixture of 32.9% hydrochloric acid solution in water that provided 0.47 moles of HCl and of 70% glycolic acid solution in water that provided 0.47 moles of glycolic acid. The contents of the dropping funnel were added dropwise to the contents of the flask. During this addition, the temperature of the liquid mixture in the flask rose because of the exothermic nature of the reaction between the amines and the acid. The mixture in the flask was then allowed to cool to room temperature. The dropping funnel was rinsed with water and recharged with 37% formaldehyde solution in water (71.9 g, 0.887 moles of formaldehyde), which was added drop-wise slowly to the flask. The reaction mixture was heated to 95° C. and the stirred reaction mixture was held there for 24 hours. (Temperatures for this first stage reaction can be 75-100° C., preferably 80-95° C.) The mixture was then cooled to 50° C. and acetone (252.0 g, 4.345 moles) was added in one portion. The dropping funnel was recharged with a second portion of 37% formaldehyde (70.3 g, 0.867 moles of formaldehyde), which was also added drop-wise. (Temperatures for this second stage reaction can be 55-65° C., preferably 60-65° C.) The solution was heated to 60° C. and held at this temperature for 24 hours.

Excess volatile materials were removed from the resulting mixture in two portions by distillation at atmospheric pressure; 0.19 kilogram of distillate were collected with the pot temperature at or below 80° C.; an additional 21 grams was removed between the temperatures of 80 and 90° C. The yield of mixed amine salts according to the invention was 0.37 kilogram. The free formaldehyde content was 0.081%.

### AMINE SALT MIXTURE SYNTHESIS EXAMPLE 1.2

This was prepared in substantially the same manner as Example 1.1, except that the acid used was 0.92 moles of glycolic acid (no hydrochloric acid). The free formaldehyde content of the product mixed amine salts was 0.071%.

The products of both of the above working examples were tested by substituting an equal mass of them for the entire content of 1,3,5-triazine, hexahydro-1,3,5-tris(2-methylphenyl)trihydrochloride, which was the only amine or amine salt present, in a commercial pickling inhibitor, then using the thus reformulated inhibitor in the same concentration as recommended for the commercial inhibitor in a 10% by volume solution of sulfuric acid in water maintained at 180° F. A cold rolled steel test panel was exposed to this inhibited sulfuric acid solution, and the weight loss from the steel test panel after 6 hours was measured in the units of pounds/square foot/day (hereinafter usually abbreviated as “#/ft²/d”). The results obtained were 0.033 for Example 1.1 and 0.047 for Example 1.2, both well below the maximum value of 0.060 specified in this test for the commercial inhibitor that was reformulated for these examples. These results are further described in Table 1 immediately below.

TABLE I

| | Glycolic Acid/ | Residual free | 6 hr test^{4,3} wt loss |
|---|---|---|---|
| Example No. | Hydrochloric Acid¹ | Formaldehyde³ | (#/ft²/day) |
| 1.1 | 1/0 | 709 | 0.047 |
| 1.2 | 0.5/0.5 | 810 | 0.033 |

¹All ratios are molar.
²Morpholine/Isopropanol amine = 0.57/0.43:: Formaldehyde/Amine = 2.0: Amine/Acetophenone = 2.4.
³QCTM 613.
⁴10%(v/v) H₂SO₄ @ 180-F
⁵Preferred value = 0.06 #/ft²/day or less

Example Group 2

### AMINE SALT MIXTURE SYNTHESIS EXAMPLE 2.1

A three necked 2000 ml round bottom flask was equipped with a mechanical stirrer, type J thermocouple attached to a temperature controller, reflux condenser, and 250 ml pressure equalized dropping funnel. The flask was charged sequentially with morpholine (87.0 g, 1.00 moles), 1-amino-2-hydroxy propane (56.3 g, 0.75 moles), acetophenone (87.6 g, 0.73 moles), and 250 ml of isopropanol solvent. The cooling water hooked up to the condenser was turned on and the dropping funnel was charged with the same concentration hydrochloric acid solution as used in Example 1.1 to provide 1.885 moles of HCl, which was added dropwise to the contents of the flask. During the addition, the temperature in the flask rose from 19 to 71° C. The flask was allowed to cool to room temperature. The dropping funnel was rinsed with water and recharged with the same concentration of formaldehyde as used in Example 1.1 to provide 2.44 moles of formaldehyde, which was added dropwise slowly to the flask. The reaction mixture was heated to 80° C. and the stirred reaction mixture was held there for 24 hours. The mixture was then cooled to 50° C. and acetone (504.0 g, 8.69 moles) was added in one portion. The dropping funnel was recharged with a second portion of the same concentration of formaldehyde solution to provide 2.385 moles of formaldehyde, which was also added dropwise. The resulting solution was heated to 60 ° C. and held at this temperature for 24 hours.

Excess volatile materials were then removed in two portions by atmospheric distillation; 0.55 kilogram of distillate were collected with the pot temperature at or below 80° C.; an additional 70 grams was removed between the temperatures of 80 and 90 ° C. The yield of mixed amine salts was 0.86 kilogram.

### AMINE SALT MIXTURE SYNTHESIS EXAMPLES 2.2 to 2.6

All of these syntheses were performed in the same manner as for Example 2.1, but the relative amounts of isopropanol amine and morpholine and the relative amounts of total amine and acetophenone were varied as noted in Table 1 below.

All of the mixed amine salt products from the Examples in Group 2 for their inhibiting effectiveness in the same manner as for Group 1. The results of these tests are also shown in Table 1.

TABLE 2

| | Molar Percent | | |
|---|---|---|---|
| | of Morpholine | | |
| Example | in Total | Molar Ratio, Total | Weight Loss, |
| Identifier | Amines | Amines:Acetophenone | #/ft²/d |
| 2.1 | 57 | 2.40 | .024 |
| 2.2 | 100 | 1.37 | .053 |
| 2.3 | 50 | 1.37 | .047 |
| 2.4 | 100 | 1.37 | .027 |
| 2.5 | 25 | 1.82 | .030 |
| 2.6 | 100 | 4.05 | .053 |

The following Tables 3 and 4 describe further embodiment of the invention in terms of the ratios of reactants (A)-(D). In Table 3 the preferred molar percentage of each reactant with respect to the total of the four reactants are set out. In Table 4 the preferred molar ratios of significant ingredients are set out.

TABLE 3
Molar Percentages of the Total

| Reactant | Preferred Range | General Range |
|---|---|---|
| Amine (C) | 8-12 | 5-15 |
| Ketone (B) | 50-60 | 40-70 |
| Formaldehyde (A) | 20-30 | 15-35 |
| Acid Catalyst (D) | 9-13 | 6-16 |

TABLE 4
Molar Ratios of Significant Reactants

| Reactant | Preferred Ratio Range | General Ratio Range |
|---|---|---|
| Amine/Acetophenone | 1.9-2.9 | 1-5 |
| Formaldehyde/Amine | 1.8-3.0 | 1.5-3.5 |
| Catalyst/Amine | 1.05-1.20 | 1-1.5 |

What is claimed is:

1. An amine salt or mixture of amine salts selected from the group consisting of secondary or tertiary amine salts, being free of but does not include an amino-aromatic ring moiety, prepared by at least one reaction among the following reactants:
(A) formaldehyde;
(B) at least one ketone conforming to the immediately following general formula 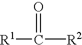
wherein each of R¹ and R², which may be the same or different, represents a monovalent organic moiety that does not include an amino-aromatic ring moiety;
(C) at least one amine constituted of molecules each of which includes at least one —N—H moiety and at least one ether or hydroxyl oxygen but does not include an amino-aromatic ring moiety; and
(D) an acid that is not part of any one of reactants (A) through (C) as described immediately above
to produce at least one secondary or tertiary amine salt reaction product in which the hydrogen atom in said at least one —N—H moiety has been replaced by a moiety having one of the two immediately following general formulas 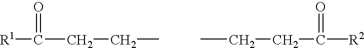
wherein each of R¹ and R² has the same meaning as in the description of reactant (B) above.

2. The amine salt or mixture of amine salts according to claim 1 wherein the ketone reactant (B) is selected from acetophenone and acetone.

3. The amine salt or mixture of amine salts according to claim 1 wherein the amine reactant (C) is selected from morpholine and 1-amino-2-hydroxy propane.

4. The amine salt or mixture of amine salts according to claim 1 wherein the acid reactant (D) is selected from glycolic acid and a mixture of glycolic acid and hydrochloric acid.

5. The amine salt or mixture of amine salts according to claim 1 wherein the acid reactant (D) is hydrochloric acid.

6. The amine salt or mixture of amine salts according to claim 1 wherein the molar percentage of the reactants (A)-(D) is
Formaldehyde (A): 15-35
Ketone (B): 40-70
Amine (C): 5-15
Acid (D): 6-16.

7. The amine salt or mixture of amine salts according to claim 1 wherein the molar percentage of the reactants (A)-(D) is
Formaldehyde (A): 20-30
Ketone (B): 50-60
Amine (C): 8-12
Acid (D): 9-13.

8. A corrosion inhibited acid pickling composition comprising an aqueous solution of an acid and an amount of the amine salt or mixture of amine salts effective to inhibit corrosion of a metal surface placed in contact with the acid pickling composition, wherein the amine salt or mixture of amine salts is selected from the group consisting of secondary or tertiary amine salts which free of an amino-aromatic ring moiety and are prepared by at least one reaction among the following reactants:
(A) formaldehyde;
(B) at least one ketone conforming to the immediately following general formula 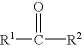
wherein each of R¹ and R², which may be the same or different, represents a monovalent organic moiety that does not include an amino-aromatic ring moiety;
(C) at least one amine constituted of molecules each of which includes at least one —N—H moiety and at least one ether or hydroxyl oxygen but does not include an amino-aromatic ring moiety; and
(D) an acid that is not part of any one of reactants (A) through (C) as described immediately above
to produce at least one secondary or tertiary amine salt reaction product in which the hydrogen atom in said at least one —N—H moiety has been replaced by a moiety having one of the two immediately following general formulas 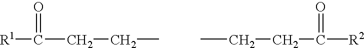
wherein each of R¹ and R² has the same meaning as in the description of reactant (B) above.

9. A corrosion inhibited acid pickling composition according to claim 8 wherein the ketone reactant (B) is selected from acetophenone and acetone.

10. A corrosion inhibited acid pickling composition according to claim 8 wherein the amine reactant (C) is selected from morpholine and 1-amino-2-hydroxy propane.

11. A corrosion inhibited acid pickling composition according to claim 8 wherein the acid reactant (D) is selected from glycolic acid and a mixture of glycolic acid and hydrochloric acid.

12. A corrosion inhibited acid pickling composition according to claim 8 wherein the acid reactant (D) is hydrochloric acid.

13. A corrosion inhibited acid pickling composition according to claim 8 wherein the molar percentage of the reactants (A)-(D) is
Formaldehyde (A): 15-35
Ketone (B): 40-70
Amine (C): 5-15
Acid (D): 6-16.

14. A corrosion inhibited acid pickling composition according to claim 8 wherein the molar percentage of the reactants (A)-(D) is
Formaldehyde (A): 20-30
Ketone (B): 50-60
Amine (C): 8-12
Acid (D): 9-13.

15. A process for reducing the corrosion of a metal surface in contact with an aqueous acid solution which comprises contacting the metal surface with an aqueous acid solution containing an amount of the amine salt or mixture of amine salts effective to inhibit corrosion of the metal surface, wherein the amine salt or mixture of amine salts is selected from the group consisting of secondary or tertiary amine salts which are free of an amino-aromatic ring moiety and are prepared by at least one reaction among the following reactants:
(A) formaldehyde;
(B) at least one ketone conforming to the immediately following general formula 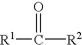
wherein each of R¹ and R², which may be the same or different, represents a monovalent organic moiety that does not include an amino-aromatic ring moiety;
(C) at least one amine constituted of molecules each of which includes at least one —N—H moiety and at least one ether or hydroxyl oxygen but does not include an amino-aromatic ring moiety; and
(D) an acid that is not part of any one of reactants (A) through (C) as described immediately above
to produce at least one secondary or tertiary amine salt reaction product in which the hydrogen atom in said at least one —N—H moiety has been replaced by a moiety having one of the two immediately following general formulas 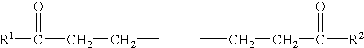
wherein each of R¹ and R² has the same meaning as in the description of reactant (B) above.

16. A process for reducing the corrosion of a metal surface according to claim 15 wherein the ketone reactant (B) for the preparation of the amine salt or mixture of amine salts is selected from acetophenone and acetone.

17. A process for reducing the corrosion of a metal surface according to claim 15 wherein the amine reactant (C) for the preparation of the amine salt or mixture of amine salts is selected from morpholine and 1-amino-2-hydroxy propane.

18. A process for reducing the corrosion of a metal surface according to claim 15 wherein the acid reactant (D) for the preparation of the amine salt or mixture of amine salts is selected from glycolic acid and a mixture of glycolic acid and hydrochloric acid.

19. A process for reducing the corrosion of a metal surface according to claim 15 wherein the acid reactant (D) for the preparation of the amine salt or mixture of amine salts is hydrochloric acid.

20. A process for reducing the corrosion of a metal surface according to claim 15 wherein the molar percentage of the reactants (A)-(D) for the preparation of the amine salt or mixture of amine salts is
Formaldehyde (A): 15-35
Ketone (B): 40-70
Amine (C): 5-15
Acid (D): 6-16.

21. A process for reducing the corrosion of a metal surface according to claim 15 wherein the molar percentage of the reactants (A)-(D) for the preparation of the amine salt or mixture of amine salts is
Formaldehyde (A): 20-30
Ketone (B): 50-60
Amine (C): 8-12
Acid (D): 9-13.

22. A process for preparing an amine salt or mixture of amine salts selected from the group consisting of secondary or tertiary amine salts which free of an amino-aromatic ring moiety, said process comprising prepared by at least one reaction among the following reactants:
(A) formaldehyde;
(B) at least one ketone conforming to the immediately following general formula 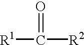
wherein each of R¹ and R² which may be the same or different, represents a monovalent organic moiety that does not include an amino-aromatic ring moiety;
(C) at least one amine constituted of molecules each of which includes at least one —N—H moiety and at least one ether or hydroxyl oxygen but does not include an amino-aromatic ring moiety; and
(D) an acid that is not part of any one of reactants (A) through (C) as described immediately above
to produce at least one secondary or tertiary amine salt reaction product in which the hydrogen atom in said at least one —N—H moiety has been replaced by a moiety having one of the two immediately following general formulas 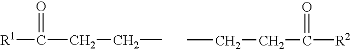
wherein each of R¹ and R² has the same meaning as in the description of reactant (B) above.

23. A process for preparing amine salt or mixture of amine salts which free of an amino-aromatic ring according to claim 22 wherein the ketone reactant (B) is selected from acetophenone and acetone.

24. A process for preparing amine salt or mixture of amine salts which free of an amino-aromatic ring according to claim 22 wherein the amine reactant (C) is selected from morpholine and 1-amino-2-hydroxy propane.

25. A process for preparing amine salt or mixture of amine salts which free of an amino-aromatic ring according to claim 22 wherein the acid reactant (D) is selected from glycolic acid and a mixture of glycolic acid and hydrochloric acid.

26. A process for preparing amine salt or mixture of amine salts which free of an amino-aromatic ring according to claim 22 wherein the acid reactant (D) is hydrochloric acid.

27. A process for preparing amine salt or mixture of amine salts which free of an amino-aromatic ring according to claim 22 wherein the molar percentage of the reactant (A)-(D) is
Formaldehyde (A): 15-35
Ketone (B): 40-70
Amine (C): 5-15
Acid (D): 6-16

28. A process for preparing amine salt or mixture of amine salts which free of an amino-aromatic ring according to claim 22 wherein the molar percentage of the reactants (A)-(D) is
Formaldehyde (A): 20-30
Ketone (B): 50-60
Amine (C): 8-12
Acid (D): 9-13
